(19) United States
(12) Patent Application Publication
(10) Pub. No.: US 2025/0276154 A1
(43) Pub. Date: Sep. 4, 2025

(54) Catheter Insertion-Tray Systems and Methods Thereof

(71) Applicant: C. R. Bard, Inc., Franklin Lakes, NJ (US)

(72) Inventor: John Gohde, Decatur, GA (US)

(21) Appl. No.: 19/199,269

(22) Filed: May 5, 2025

Related U.S. Application Data
(63) Continuation of application No. 18/536,972, filed on Dec. 12, 2023, now Pat. No. 12,290,645.
Application No. 18/536,972 is a continuation of application No. 16/497,770, filed on Sep. 25, 2019, now Pat. No. 11,896,778.
(60) Provisional application No. 62/479,687, filed on Mar. 31, 2017.

(51) Int. Cl. A61M 25/01; A61B 42/10; A61B 46/00; A61B 46/23; A61M 25/00

(52) U.S. Cl. CPC A61M 25/0111; A61B 42/10; A61B 46/23; A61B 46/30; A61M 25/0017; A61M 25/002; A61B 2046/234; A61M 2207/00; A61M 2210/1085

(57) ABSTRACT

A catheterization tray includes a sterile side configured to enable a user to carry out sterile steps of a catheterization procedure in a sterile field and a set of sterile items positioned in the sterile side of the catheterization tray. The catheterization tray further includes a non-sterile side configured to enable a user to carry out non-sterile steps of the catheterization procedure outside of the sterile field and a set of non-sterile items positioned in the non-sterile side of the catheterization tray. The catheterization tray further includes a partition extending between the sterile side and the non-sterile side from a first side wall of the catheterization tray to a second side wall of the catheterization tray to maintain sterility of the sterile side of the catheterization tray.

### PRIORITY

This application is a continuation of U.S. patent application Ser. No. 18/536,972, filed Dec. 12, 2023, now U.S. Pat. No. 12,290,645, which is a continuation of U.S. patent application Ser. No. 16/497,770, filed Sep. 25, 2019, now U.S. Pat. No. 11,896,778, which is a U.S. national stage of International Application No. PCT/US2018/025260, filed Mar. 29, 2018, which claims the benefit of priority to U.S. Provisional Application No. 62/479,687, filed Mar. 31, 2017, each of which is incorporated by reference in its entirety into this application.

### BACKGROUND

Catheterization packages including catheter-insertion trays are generally designed and optimized for a catheter-insertion process by one person such as a nurse, wherein the nurse stands on one side of a patient's bed and performs all preparation and insertion steps of the catheter-insertion process from one side of a catheter-insertion tray. By the very nature of the one-person catheter-insertion process, the nurse can introduce unwanted contamination to sterile parts of the catheter-insertion tray and cause unintended complications subsequent to the catheter-insertion process. As such, the catheter-insertion process can benefit from certain measures to ensure a sterile technique. Provided herein in some embodiments are systems and methods that address the foregoing.

### SUMMARY

Provided herein in some embodiments is a catheterization package including a catheterization tray and contents for a catheterization procedure. The catheterization tray can include a structural configuration for maintaining a sterile field about a patient throughout the catheterization procedure, the sterile field including at least a portion of the tray. The structural configuration of the tray can provide a sterile side of the tray designated for a first person performing sterile steps of the catheterization procedure in the sterile field. The structural configuration of the tray can also provide a non-sterile side of the tray designated for either the first person or a second person performing non-sterile steps of the catheterization procedure outside the sterile field. The contents for the catheterization procedure can include a perineal care kit, two or more pairs of gloves, and a drainage system including a catheter.

Also provided herein in some embodiments is a catheterization package including a catheterization tray and contents for a catheterization procedure. The catheterization tray can include a structural configuration for maintaining a sterile field about a patient throughout the catheterization procedure, the sterile field including at least a portion of the tray. The structural configuration of the tray can provide a sterile side of the tray designated for a first person performing sterile steps of the catheterization procedure in the sterile field. The structural configuration of the tray can also provide a non-sterile side of the tray designated for either the first person or a second person performing non-sterile steps of the catheterization procedure outside the sterile field. The structural configuration can include at least a partition separating the sterile and non-sterile sides of the tray along a length of the tray in a medial portion of the tray. The contents for the catheterization procedure can include a perineal care kit configured for cleaning a patient's peri-urethral area, two or more pairs of sterile gloves, and a drainage system. The drainage system can include a catheter, including a drainage catheter, such as a urinary catheter, for example, a Foley catheter; drainage tubing; and a drainage bag configured for draining material such as urine from a patient.

Also provided herein in some embodiments is a method for a catheterization package including molding a catheterization tray for the catheterization package. The catheterization tray can include a structural configuration for maintaining a sterile field about a patient throughout a catheterization procedure, the sterile field including at least a portion of the tray. The structural configuration of the tray can provide a sterile side of the tray designated for a first person performing sterile steps of the catheterization procedure in the sterile field. The structural configuration of the tray can also provide a non-sterile side of the tray designated for either the first person or a second person performing non-sterile steps of the catheterization procedure outside the sterile field.

Also provided herein in some embodiments is a method of a catheterization package including unwrapping the catheterization package to reveal a catheterization tray, removing a first set of contents for a catheterization procedure from a non-sterile side of the tray, and removing a second set of contents for the catheterization procedure from a sterile side of the tray. Unwrapping the catheterization package to reveal the catheterization tray can include revealing a structural configuration of the tray for maintaining a sterile field about a patient throughout a catheterization procedure, the sterile field including at least a portion of the tray. The structural configuration of the tray can include a sterile side of the tray designated for a first person performing sterile steps of the catheterization procedure in the sterile field. The structural configuration of the tray can also provide a non-sterile side of the tray designated for either the first person or a second person performing non-sterile steps of the catheterization procedure outside the sterile field. Removing the first set of contents for the catheterization procedure from the non-sterile side of the tray can include removing an underpad and a fenestrated drape from the non-sterile side of the catheterization tray. Removing the second set of contents for the catheterization procedure from the sterile side of the tray can include removing a drainage system including a urinary catheter, drainage tubing, and a drainage bag from the sterile side of the catheterization tray.

These and other features of the concepts provided herein may be better understood with reference to the drawings, description, and appended claims.

### BRIEF DESCRIPTION OF DRAWINGS

FIG. 1 provides a schematic illustrating a first layer of a catheterization package for use by one or more persons in accordance with some embodiments.

FIG. 2 provides a schematic illustrating a second layer of the catheterization package of FIG. 1 in accordance with some embodiments.

FIG. 3 provides a schematic illustrating a third layer of the catheterization package of FIG. 1 in accordance with some embodiments.

FIG. 4 provides a schematic illustrating a first layer of a one-person catheterization package.

FIG. 5 provides a schematic illustrating a second layer of the one-person catheterization package of FIG. 4.

FIG. 6 provides a schematic illustrating a third layer of the one-person catheterization package of FIG. 4.

### DETAILED DESCRIPTION

Before some particular embodiments are provided in greater detail, it should be understood that the particular embodiments provided herein do not limit the scope of the concepts provided herein. It should also be understood that a particular embodiment provided herein can have features that can be readily separated from the particular embodiment and optionally combined with or substituted for features of any of a number of other embodiments provided herein.

Regarding terminology used herein, it should also be understood the terminology is for the purpose of describing some particular embodiments, and the terminology does not limit the scope of the concepts provided herein. Unless indicated otherwise, ordinal numbers (e.g., first, second, third, etc.) are used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. It should also be understood that, unless indicated otherwise, any labels such as “left,” “right,” “front,” “back,” “top,” “bottom,” “forward,” “reverse,” “clockwise,” “counter clockwise,” “up,” “down,” or other similar terms such as “upper,” “lower,” “aft,” “fore,” “vertical,” “horizontal,” “proximal,” “distal,” and the like are used for convenience and are not intended to imply, for example, any particular fixed location, orientation, or direction. Instead, such labels are used to reflect, for example, relative location, orientation, or direction. It should also be understood that the singular forms of “a,” “an,” and “the” include plural references unless the context clearly dictates otherwise.

Unless defined otherwise, all technical and scientific terms used herein have the same meaning as commonly understood by those of ordinary skill in the art.

Catheterization packages including catheter-insertion trays are generally designed and optimized for a catheter-insertion process by one person such as a nurse, wherein the nurse stands on one side of a patient's bed and performs all preparation and insertion steps of the catheter-insertion process from one side of a catheter-insertion tray. By the very nature of the one-person catheter-insertion process, the nurse can introduce unwanted contamination to sterile parts of the catheter-insertion tray and cause unintended complications subsequent to the catheter-insertion process.

Sterile technique in the catheter-insertion process can be compromised when previously sterile, gloved hands having touched a non-sterile patient cross above a sterile surface, re-enter a sterile field, or touch sterile components such as a sterile catheter. For example, sterile technique can be compromised when sterile-gloved hands approach or touch a patient during perineal care (“pericare”), draping, or cleansing, each of which can require patient retraction or positioning (e.g., moving skin, genitalia, etc. into proper position so catheterization is possible). It can be difficult to eliminate such sterile technique-compromising activities because these activities are often necessary to a one-person catheter-insertion process with existing one-person catheterization packages—not to mention such compromises in sterile technique often go unnoticed.

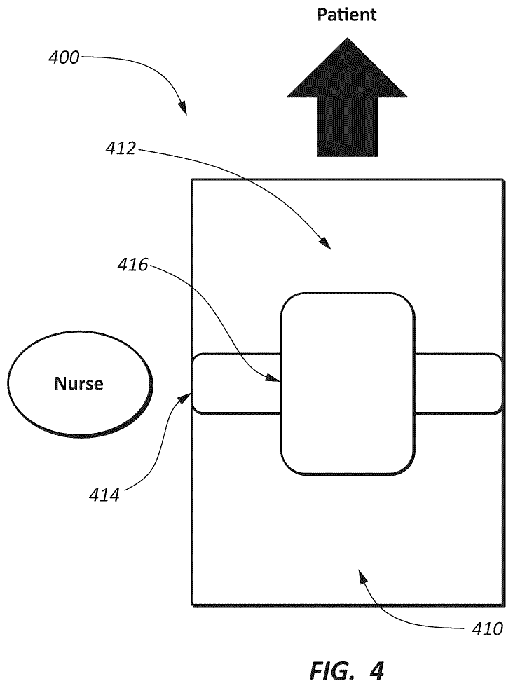

FIG. 4 provides a schematic illustrating a first layer 410 of a one-person catheterization package 400.

As shown, the catheterization package 400 includes the first layer 410 including a wrap 412, a belly band 414 around the wrap 412, and a perineal care kit 416. The one-person catheterization package 400 is designed such that one nurse starts a catheterization procedure on a patient from one side of the catheterization package 400 or a catheterization tray thereof, which is typically biased to right-handed nurses. Clean gloves from a wall or elsewhere in a room are donned for the catheterization procedure and a perineal-urethral cleansing of the patient is subsequently performed. After the cleansing, the gloves are removed, and the nurse uses hand sanitizer to clean his or her hands prior to unwrapping the catheterization package 400.

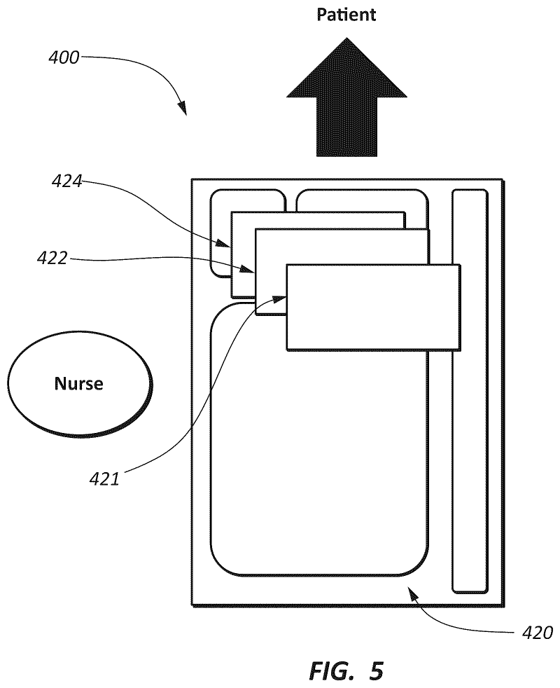

FIG. 5 provides a schematic illustrating a second layer 420 of the one-person catheterization package 400 of FIG. 4.

As shown, the catheterization package 400 includes the second layer 420 including a pair of gloves 421, an underpad 422, and a fenestrated drape 424 upon unwrapping the catheterization package 400. Once unwrapped, a sterile portion of the catheterization package 400 is exposed and, therefore, sterile gloves are donned. Continuing with the catheterization procedure, the nurse places the underpad 422 under the patient followed by the fenestrated drape 424 over the patient, each of which carries with it a contamination risk from manipulating the patient.

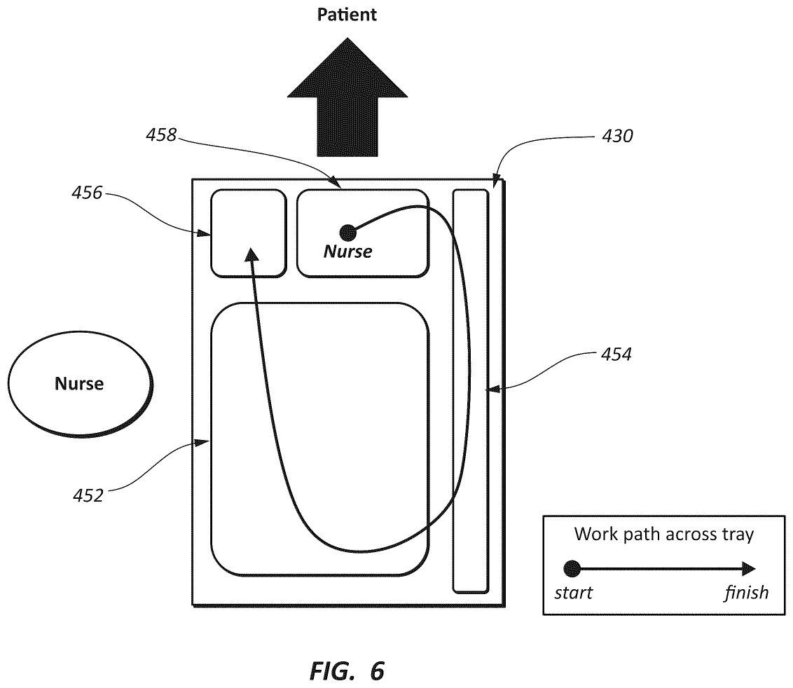

FIG. 6 provides a schematic illustrating a third layer 430 of the one-person catheterization package 400 of FIG. 4.

As shown, the catheterization package 400 includes the third layer 430 including a catheter assembly 452, syringes 454, a sample container 456, and a preparative solution 458 subsequent to removing contents of the second layer 420. Continuing with the catheterization procedure, the nurse cleans the patient with the preparation solution 458, which further carries with it a contamination risk from manipulating the patient. Subsequently, the nurse prepares a catheter of the catheter assembly with lubricant and water. Note that this requires the nurse to move potentially contaminated hands across a sterile field above the catheter assembly, as well as even touch the catheter with contaminated hands. The nurses potentially contaminating work path is shown in FIG. 6 by an arrow starting from a section of the tray including the preparative solution 458 to a section of the tray including the sample container 456.

In view of the foregoing, the catheter-insertion process can benefit from certain measures to ensure a sterile technique in the catheter-insertion process. For example, the catheter-insertion process can benefit from more than one person or health care provider to ensure a sterile technique in the catheter-insertion process. Provided herein in some embodiments are a catheterization package and a catheterization procedure thereof designed to ensure a sterile technique in the catheter-insertion process. In some embodiments, the catheterization package and a catheterization procedure thereof are designed for catheter insertion by more than one person or health care provider. The present disclosure, however, is also applicable to catheter insertion by a single healthcare provider, such as a nurse.

For example, in some embodiments is a catheterization package is provided including a catheterization tray and contents for a catheterization procedure. The catheterization tray can include a structural configuration for maintaining a sterile field about a patient throughout the catheterization procedure, the sterile field including at least a portion of the tray. The structural configuration of the tray can provide a sterile side of the tray designated for a first person performing sterile steps of the catheterization procedure in the sterile field. The structural configuration of the tray can also provide a non-sterile side of the tray designated for either the first person or a second person performing non-sterile steps of the catheterization procedure outside the sterile field. The contents for the catheterization procedure can include a perineal care kit, two or more pairs of gloves, and a drainage system including a catheter.

Such a catheterization package and catheterization tray can facilitate a sterile catheterization procedure by isolating sterile contents of the catheterization package and steps of the catheterization procedure from previously sterile contents of the catheterization package and non-sterile steps of the catheterization procedure, thereby reducing or eliminating compromises in sterile technique.

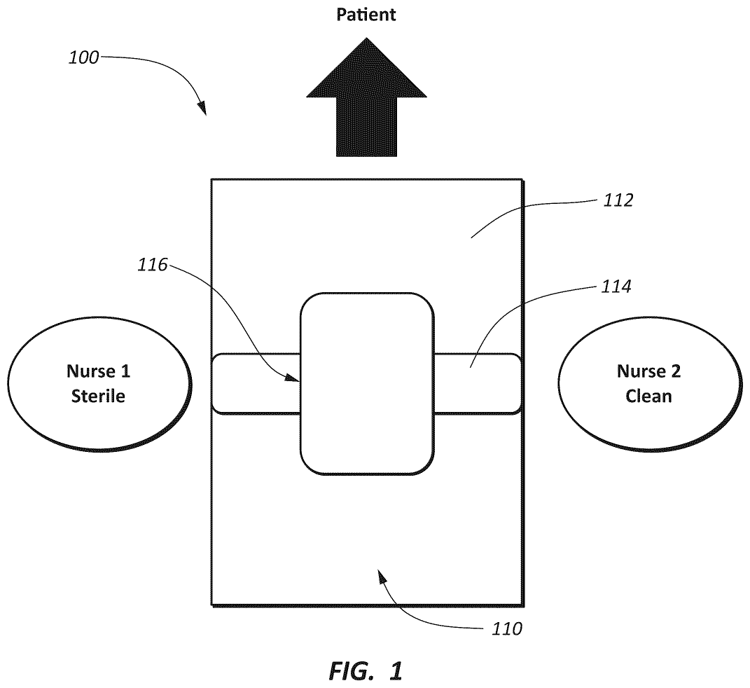

FIG. 1 provides a schematic illustrating a first layer 110 of a catheterization package 100 for use by one or more persons in accordance with some embodiments.

As shown, the catheterization package 100 can include the first layer 110 a sterile wrap 112, a belly band 114 configured to hold the sterile wrap, and a perineal care kit 116 including hand sanitizer, and, optionally, two or more pairs of gloves.

The sterile wrap can be a central supply room (“CSR”) wrap and the belly band 114 can be configured to hold the sterile wrap around the catheterization tray. The perineal care kit 116 can be located outside the sterile wrap optionally with at least one package of hand sanitizer. A first pair of gloves of the two or more pairs of gloves can be located outside the sterile wrap, or the first pair of gloves of the two or more pairs of gloves can be located inside the sterile wrap. Alternatively, a first pair of gloves of the two or more pairs of gloves can be located outside the sterile wrap and a second pair of gloves of the two or more pairs of gloves can be located inside the sterile wrap.

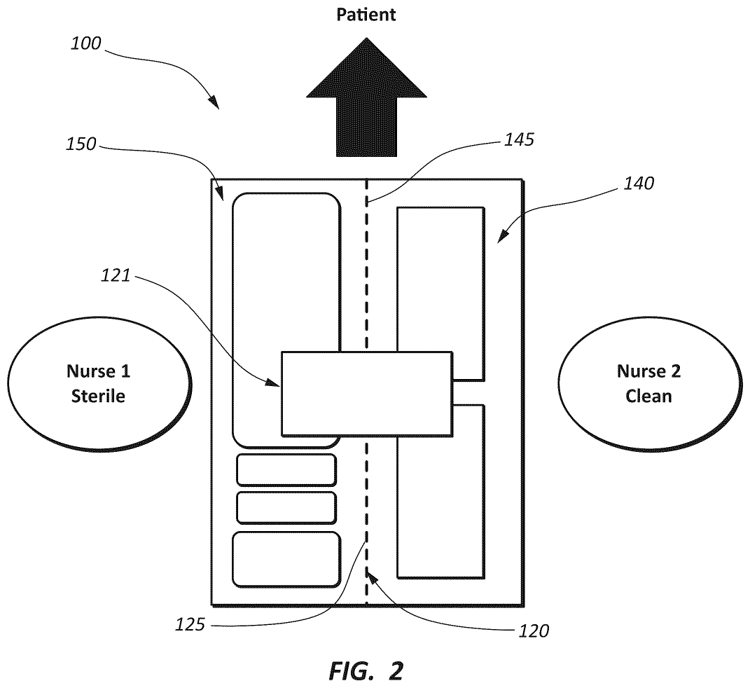

FIG. 2 provides a schematic illustrating a second layer 120 of the catheterization package 100 of FIG. 1 in accordance with some embodiments.

As shown, the catheterization package 100 can include the second layer 120 including a catheterization tray, optionally, two or more pairs of gloves 121 if not part of the first layer 110 or in addition thereto, a clean or non-sterile side 140 of the tray, and a sterile side 150 of the tray.

It should be understood that while the non-sterile side 140 of the tray is referred to herein as “non-sterile,” “non-sterile” is intended to refer to a particular side of the tray that need not remain sterile or is otherwise outside the sterile field during the catheterization process. Contents of the catheterization package 100 on the non-sterile side 140 can be sterile as packaged in the catheterization package 100.

The tray can have a structural configuration including at least a partition 125 separating the sterile and non-sterile sides of the tray along a length of the tray in a medial portion of the tray; however, the structural configuration of the tray is not limited thereto for separating the sterile and non-sterile sides of the tray. The structural configuration of the tray can also be such that the contents in the tray can be layered in the tray in step with the sterile and the non-sterile steps of the catheterization procedure.

The tray can also include instructions for the catheterization procedure imprinted directly on the tray, at least some of which instructions are revealed in step with the sterile and the non-sterile steps of the catheterization procedure as the contents in the tray are removed. Because the structural configuration of the tray accommodates layered contents for the catheterization procedure, the tray is also configured such that removal of the contents from the tray can reveal the instructions (e.g., the instructions imprinted on the tray), additional contents for the catheterization procedure, or a combination of the instructions and the additional contents in step with the sterile and the non-sterile steps of the catheterization procedure.

The tray can be configured with a bias toward right-handed or left-handed catheter insertion by swapping the non-sterile side 140 of the tray and the sterile side 150 of the tray. For example, the tray of FIG. 2 can accommodate a left-handed catheter insertion, but by swapping the non-sterile side 140 of the tray and the sterile side 150 of the tray, the tray can accommodate a right-handed catheter insertion.

The tray can be of any size necessary for a catheterization procedure including those requiring multiple drapes or even walleted drapes.

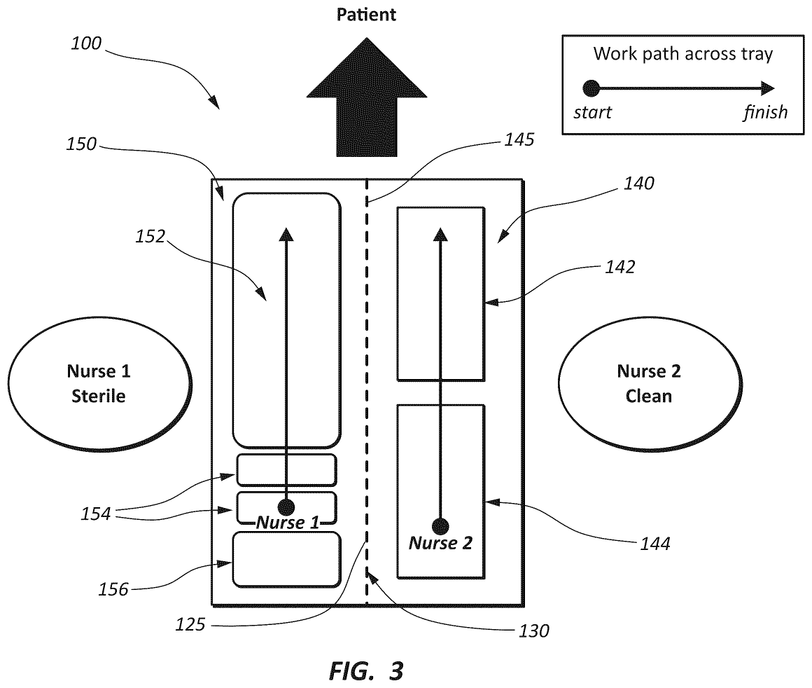

FIG. 3 provides a schematic illustrating a third layer 130 of the catheterization package 100 of FIG. 1 in accordance with some embodiments.

As shown, the catheterization package 100 can include the third layer 130 including the non-sterile side 140 of the tray with an underpad 142 and a fenestrated drape 144. The third layer 130 of the catheterization package 100 can further include the sterile side 150 of the tray with a catheter assembly or drainage system 152, syringes 154, and a sample container 156. The arrow for Nurse 1 and the arrow for Nurse 2 show work paths for how contents of the catheterization package 100 can be removed without compromising sterile technique and the sterile field of which the sterile side 150 of the tray is part. It should be understood that the arrows for Nurses 1 and 2 merely show work paths across the catheterization package 100. Nurse 1 and Nurse 2 need not reflect different nurses. Nurse 1 and Nurse 2 can be the same nurse at different times or different nurses at the same or different times.

With respect to the non-sterile side 140 of the tray, the tray can further include a skin-preparation kit including a package of an antiseptic (e.g., an iodophor such as iodopovidone, tincture of iodine, aqueous iodine, etc.) and one or more swabs configured for preparing a patient for inserting a catheter of the catheter assembly.

With respect to the sterile side 150 of the tray, the drainage system 152 thereof can include a catheter, including a drainage catheter, such as a urinary catheter, for example, a Foley catheter; drainage tubing; and a drainage bag configured for draining material such as urine from a patient. For example, the drainage system 152 can be configured for draining urine from a patient's bladder and include a Foley catheter, drainage tubing, and a drainage bag. At least one syringe of the syringes 154 can include a syringe of sterile saline for inflating a balloon of a Foley catheter, and at least one other syringe of the syringes 154 can include a syringe (or another dispensing container) containing lubricant for lubricating the Foley catheter in accordance with the catheterization procedure.

In accordance with FIGS. 1-3, a catheterization package can include, in some embodiments, a catheterization tray and contents for a catheterization procedure. The catheterization tray can include a structural configuration for maintaining a sterile field about a patient throughout the catheterization procedure, the sterile field including at least a portion of the tray. The structural configuration of the tray can provide a sterile side of the tray designated for a first person performing sterile steps of the catheterization procedure in the sterile field. The structural configuration of the tray can also provide a non-sterile side of the tray designated for either the first person or a second person performing non-sterile steps of the catheterization procedure outside the sterile field. The contents for the catheterization procedure can include a perineal care kit, two or more pairs of gloves, and a drainage system including a catheter.

In such embodiments of the catheterization package, the structural configuration of the tray can include at least a partition (e.g., a partition molded into the tray itself) separating the sterile and non-sterile sides of the tray along a length of the tray in a medial portion of the tray; however, the structural configuration of the tray is not limited thereto for separating the sterile and non-sterile sides of the tray. The structural configuration of the tray can also be such that the contents in the tray for catheterization procedure can be layered to reveal the instructions, additional contents for catheterization procedure, or a combination of the instructions and the additional contents in step with the sterile and the non-sterile steps of the catheterization procedure. The tray can also include instructions for the catheterization procedure imprinted directly on the tray, at least some of which instructions are revealed in step with the sterile and the non-sterile steps of the catheterization procedure as the contents in the tray are removed.

Further in accordance with FIGS. 1-3, a catheterization package can include, in some embodiments, a catheterization tray and contents for a catheterization procedure. The catheterization tray can include a structural configuration for maintaining a sterile field about a patient throughout the catheterization procedure, the sterile field including at least a portion of the tray. The structural configuration of the tray can provide a sterile side of the tray designated for a first person performing sterile steps of the catheterization procedure in the sterile field. The structural configuration of the tray can also provide a non-sterile side of the tray designated for the first person or a second person performing non-sterile steps of the catheterization procedure outside the sterile field. The structural configuration can include at least a partition (e.g., a partition molded into the tray itself) separating the sterile and non-sterile sides of the tray along a length of the tray in a medial portion of the tray. The contents for the catheterization procedure can include a perineal care kit configured for cleaning a patient's peri-urethral area, two or more pairs of sterile gloves, and a drainage system. As provided herein, the drainage system can include a Foley catheter, drainage tubing, and a drainage bag configured for draining urine from a patient's bladder.

In such embodiments of the catheterization package, the contents for the catheterization procedure can further include a syringe of sterile saline configured for inflating a balloon of the Foley catheter, a container (e.g., syringe) containing lubricant configured for lubricating the Foley catheter, and a sterile sample container configured for holding a patient's urine sample. The sterile side of the tray can be configured to include the drainage system, the syringe of sterile saline, the container containing lubricant, and the sample container.

In such embodiments of the catheterization package, the contents for the catheterization procedure can further include a waterproof underpad configured for placement under a patient and a fenestrated drape configured for placement over a patient. The non-sterile side of the tray can be configured to include the underpad and the fenestrated drape.

In such embodiments of the catheterization package, the contents for the catheterization procedure can further include a skin-preparation kit including a package of an antiseptic (e.g., an iodophor such as iodopovidone, tincture of iodine, aqueous iodine, etc.) and one or more swabs configured for preparing a patient for inserting the Foley catheter. The non-sterile side of the tray is configured to include the skin-preparation kit.

In such embodiments of the catheterization package, the tray can include instructions for the catheterization procedure imprinted directly on the tray, at least some of which instructions are revealed in step with the sterile and the non-sterile steps of the catheterization procedure as the contents in the tray are removed. The structural configuration of the tray can be such that the contents in the tray can be layered to reveal the instructions, additional contents, or a combination of the instructions and the additional contents in step with the sterile and the non-sterile steps of the catheterization procedure.

In such embodiments of the catheterization package, the catheterization package can further comprise a sterile wrap and a belly band configured to hold the sterile wrap around the catheterization tray. The perineal care kit can be located outside the sterile wrap with a package of hand sanitizer. In addition, a first pair of gloves of the two or more pairs of gloves can be located outside the sterile wrap. Additionally or alternatively, a second pair of gloves of the two or more pairs of gloves can be located inside the sterile wrap.

### Manufacturing

A catheterization package such at the catheterization package 100 of FIGS. 1-3 can be manufactured by initially creating a catheterization tray for the catheterization package and subsequently filling the tray with contents for a catheterization procedure.

With respect to creating the tray, the tray can be created by molding any of a number of moldable materials into a form of the tray. Again, the catheterization tray can include a structural configuration for maintaining a sterile field about a patient throughout the catheterization procedure, the sterile field including at least a portion of the tray. The structural configuration of the tray can provide a sterile side of the tray designated for a first person performing sterile steps of the catheterization procedure in the sterile field. The structural configuration of the tray can also provide a non-sterile side of the tray designated for either the first person or a second person performing non-sterile steps of the catheterization procedure outside the sterile field. For example, the structural configuration of the tray can include at least a partition separating the sterile and non-sterile sides of the tray along a length of the tray in a medial portion of the tray. A tray with such a structural configuration can be created by molding any of the number of moldable materials in a molding process selected from, but not limited to, compression molding, injection molding, thermoforming, and a combination thereof.

With respect to filling the tray with contents for the catheterization procedure, the tray can be filled by placing an underpad and a fenestrated drape in the non-sterile side of the catheterization tray. The tray can be further filled by placing a drainage system in the sterile side of the catheterization tray, the drainage system including, for example, a Foley catheter, drainage tubing, and a drainage bag.

Subsequent to creating the tray and filling the tray with contents for the catheterization procedure, the tray can be wrapped in a sterile wrap. A belly band can be added to hold the sterile wrap around the catheterization tray. Lastly, a perineal care kit and a package of hand sanitizer can be added outside the sterile wrap resulting in a catheterization package.

### Use

A catheterization package such as the catheterization package 100 of FIGS. 1-3 can be used by one or more persons such as by one or more nurses (e.g., two nurses) for one or more steps of a catheterization procedure.

A catheterization procedure using the catheterization package 100 of FIGS. 1-3 can include unwrapping the catheterization package to reveal a catheterization tray, removing a first set of contents for the catheterization procedure from a non-sterile side of the tray, and removing a second set of contents for the catheterization procedure from a sterile side of the tray. Unwrapping the catheterization package to reveal the catheterization tray can include revealing a structural configuration of the tray for maintaining a sterile field about a patient throughout a catheterization procedure, the sterile field including at least a portion of the tray. Again, the structural configuration of the tray can include a sterile side of the tray and a non-sterile side of the tray, wherein the sterile side of the tray is designated for a first person performing sterile steps of the catheterization procedure in the sterile field, and wherein the non-sterile side of the tray is designated for either the first person or a second person performing non-sterile steps of the catheterization procedure outside the sterile field. Removing the first set of contents for the catheterization procedure from the non-sterile side of the tray can include removing an underpad and a fenestrated drape from the non-sterile side of the catheterization tray. (See work path of Nurse 2 in FIG. 3, wherein Nurse 2 removes the first set of contents from the tray.) Removing the second set of contents for the catheterization procedure from the sterile side of the tray can include removing a drainage system including, for example, a Foley catheter, drainage tubing, and a drainage bag from the sterile side of the catheterization tray. (See work path of Nurse 1 in FIG. 3, wherein Nurse 1 removes the second set of contents from the tray.)

The catheterization procedure can further include removing a third set of contents for the catheterization procedure from an outside of the catheterization package prior to unwrapping the catheterization package. Removing the third set of contents for the catheterization procedure from the catheterization package can include removing a perineal care kit and a package of hand sanitizer from outside a sterile wrap wrapping the catheterization package prior to unwrapping the catheterization package. The configuration of the catheterization package enables performance of the non-sterile step of removing the perineal care kit and the hand sanitizer from the catheterization package while maintaining the sterile field.

The catheterization procedure can further include placing the underpad under the patient, cleaning a peri-urethral area of the patient with the perineal care kit, and placing the fenestrated drape over the patient. The structural configuration of the tray enables performance of the non-sterile steps of placing the underpad under the patient, cleaning the peri-urethral area of the patient with the perineal care kit, and placing the fenestrated drape over the patient by the second person while maintaining the sterile field. One example of a suitable perineal care kit that can be used in accordance with the present disclosure is described in PCT Publication No. WO 2016/126555, the disclosure of which is incorporated herein by reference in its entirety.

The catheterization procedure can further include preparing the patient and the catheter for catheterization and subsequently catheterizing the patient. Preparing the patient for catheterization can include preparing the patient with a skin-preparation kit of the catheterization package, the skin-preparation kit including an antiseptic (e.g., an iodophor such as iodopovidone, tincture of iodine, aqueous iodine, etc.) and on or more swabs. Preparing the catheter for catheterization can include lubricating the catheter with a lubricant from a container containing the lubricant from the catheterization package. The structural configuration of the tray enables performance of the sterile steps of removing the drainage system including the catheter from the catheterization tray, preparing the patient and the catheter for catheterization, and catheterizing the patient by the first person while maintaining the sterile field.

In view of the foregoing, the catheterization package 100 of FIGS. 1-3 and the catheterization tray thereof set up two fields including one sterile field and one non-sterile field such that at least one person designated as a sterile nurse and at least one other person designated as a non-sterile nurse (optionally, the sterile nurse at a different time) can respectively ensure non-compromising sterile technique in a catheter-insertion process. The sterile nurse's responsibility is to manage the sterile parts of the catheterization package as well as insertion of the catheter. The non-sterile nurse's responsibility is to manage the non-sterile components (e.g., previously sterile components that need not remain sterile) of the catheter-insertion process as well some patient preparation (e.g., cleaning the peri-urethral area of the patient with the perineal care kit) and draping. As such, the non-sterile nurse cleans, touches, and positions the patient while the sterile nurse maintains the sterile field and inserts the catheter with sterile-gloved hands that have not been compromised.

The configuration of the catheterization package and the structural configuration of the catheterization tray facilitate the roles of the sterile nurse and the non-sterile nurse. As shown in FIGS. 1-3, the tray is designed to be used from two separate sides with appropriate contents for the catheterization procedure on each side of the two separate sides to be accessed by the appropriate person (e.g., the sterile nurse and the non-sterile nurse or one nurse acting as both the sterile nurse and the non-sterile nurse at different times). For example, the perineal care kit (if not packaged outside the catheterization package), one or more pairs of gloves, and the skin-preparation kit (e.g., the antiseptic and swabs) can be located on the non-sterile side of the tray while one or more other pairs of gloves and the drainage system including the catheter can be located on the sterile side of the tray. The non-sterile nurse on the right-hand side of the catheterization package 100 of FIG. 1 is designated for cleaning a patient's peri-urethral area with gloves (e.g., the gloves from first layer 110 or second layer 120). If two nurses, both nurses subsequently remove their gloves and clean their hands with the hand sanitizer, optionally packaged in individualized packages of hand sanitizer. In accordance with FIG. 3, the sterile nurse proceeds to remove the drainage system from the sterile side of the tray and prepare the catheter with lubricant and water with sterile-gloved hands. Meanwhile, the non-sterile nurse proceeds to place the underpad, place the fenestrated drape, and apply the antiseptic (e.g., iodopovidone) with the swabs to clean the patient. The non-sterile nurse can then manipulate the patient as required for catheter insertion, after which the sterile nurse can insert the catheter. Note that this does not require any potentially contaminated hands to cross over into the sterile field or touch sterile contents for the catheterization procedure located in the sterile field.

While some particular embodiments have been provided herein, and while the particular embodiments have been provided in some detail, it is not the intention for the particular embodiments to limit the scope of the concepts presented herein. Additional adaptations and/or modifications can appear to those of ordinary skill in the art, and, in broader aspects, these adaptations and/or modifications are encompassed as well. Accordingly, departures may be made from the particular embodiments provided herein without departing from the scope of the concepts provided herein.

What is claimed is:

1. A catheterization tray, comprising:
a sterile side configured to enable a user to carry out sterile steps of a catheterization procedure in a sterile field;
a set of sterile items positioned in the sterile side;
a non-sterile side configured to enable a user to carry out non-sterile steps of the catheterization procedure outside of the sterile field;
a set of non-sterile items positioned in the non-sterile side; and
a partition extending between the sterile side and the non-sterile side from a first side wall of the catheterization tray to a second side wall of the catheterization tray to maintain sterility of the sterile side.

2. The catheterization tray according to claim 1, wherein the set of sterile items are ordered in accordance with a sterile work path.

3. The catheterization tray according to claim 1, wherein the set of non-sterile items are ordered in accordance with a non-sterile work path.

4. The catheterization tray according to claim 1, further comprising a sterile wrap and a belly band configured to hold the sterile wrap around the catheterization tray.

5. The catheterization tray according to claim 4, further comprising a perineal care kit positioned outside of the sterile wrap along with a package of hand sanitizer.

6. The catheterization tray according to claim 4, further comprising at least a first pair of gloves positioned outside of the sterile wrap.

7. The catheterization tray according to claim 4, further comprising at least a first pair of gloves positioned inside of the sterile wrap.

8. The catheterization tray according to claim 1, wherein the set of sterile items include a drainage system and a catheter.

9. The catheterization tray according to claim 8, wherein the set of sterile items further include a syringe of sterile saline, a container containing lubricant, and a sterile sample container.

10. The catheterization tray according to claim 1, wherein the set of non-sterile items include a waterproof underpad configured for placement under a patient and a fenestrated drape configured for placement over the patient.

11. The catheterization tray according to claim 10, wherein the set of non-sterile items further include a skin-preparation kit including a package of an antiseptic and one or more swabs configured for preparing the patient for inserting a urinary catheter.

12. The catheterization tray according to claim 1, further comprising instructions for the catheterization procedure imprinted directly on a surface of the catheterization tray, the instructions revealed in accordance with the sterile steps and the non-sterile steps of the catheterization procedure as the set of sterile items and the set of non-sterile items are removed from the catheterization tray.

13. A method of using a catheterization package, comprising:
unwrapping the catheterization package to reveal a catheterization tray, the catheterization tray comprising:
a sterile side configured to enable a user to carry out sterile steps of a catheterization procedure in a sterile field;
a non-sterile side configured to enable a user to carry out non-sterile steps of the catheterization procedure outside of the sterile field; and
a partition extending between the sterile side and the non-sterile side from a first side wall of the catheterization tray to a second side wall of the catheterization tray to maintain sterility of the sterile side of the catheterization tray;
removing an underpad and a fenestrated drape from the non-sterile side of the catheterization tray; and
removing a drainage system including a urinary catheter, drainage tubing, and a drainage bag from the sterile side of the catheterization tray.

14. The method according to claim 13, further comprising:
removing a perineal care kit and a package of hand sanitizer from outside a sterile wrap of the catheterization package; and
cleaning a peri-urethral area of a patient with the perineal care kit.

15. The method according to claim 13, further comprising:
placing the underpad under a patient; and
placing the fenestrated drape over the patient while maintaining the sterile field.

16. The method according to claim 13, further comprising:
preparing a patient and the urinary catheter for catheterization; and
catheterizing the patient while maintaining the sterile field.
